(19) United States
(12) Patent Application Publication
(10) Pub. No.: US 2007/0203240 A1
(43) Pub. Date: Aug. 30, 2007

(54) Personal care compositions and methods for regulating mammalian hair growth

(75) Inventors: John Erich Oblong, Loveland, OH (US); Ben Charlton Hulette, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/711,332

(22) Filed: Feb. 27, 2007

Related U.S. Application Data
(60) Provisional application No. 60/777,068, filed on Feb. 27, 2006.

(51) Int. Cl. A61K 31/195; A61K 31/045; A61K 31/14; A61K 31/155

(52) U.S. Cl. 514/561; 514/631; 514/642; 514/724

(57) ABSTRACT

Personal care composition, comprising a first ingredient selected from the group consisting of α-phenyl butyl nitrone (PBN), PBN doxylcyclohexane radicals, 5,5-dimethyl pyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, spin-trapping derivatives thereof, and mixtures thereof, a second ingredient selected from the group consisting of particulate materials, panthenol, pantothenic acid derivatives, tanning actives, and mixtures thereof; and a dermatologically acceptable carrier.

### CROSS REFERENCE TO RELATED APPLICATION

This application claims the benefit of U.S. Provisional Application No. 60/777,068, filed Feb. 27, 2006.

### FIELD

The present invention relates to personal care compositions containing a nitrone derivative for regulating the condition of keratinous tissue, including, but not limited to, regulating mammalian hair growth.

### BACKGROUND

Peter Procter, in U.S. Pat. No. 5,723,502, discloses the use of topical nitrone and nitroso spin traps, such as N-t-butyl-.alpha-phenylnitrone (PBN), for treating hair loss. That is, it is known to employ nitrone derivatives to stimulate hair growth.

In direct contrast to the above disclosure, Applicant has unexpectedly discovered that nitrone derivatives are useful for retarding, inhibiting, or eliminating hair growth, or otherwise regulating mammalian hair growth.

### DETAILED DESCRIPTION

The present invention may be understood more readily by reference to the following detailed description of illustrative and preferred embodiments. It is to be understood that the scope of the claims is not limited to the specific ingredients, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values forms another embodiment. All ranges are inclusive and combinable.

All percentages, parts and ratios are based upon the total weight of the personal care compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

As used herein, the term “personal care compositions” are those used to treat or care for, or somehow moisturize, improve, or clean keratinous tissue. Products contemplated by the phrase “personal care composition” include, but are not limited to, lotions, creams, moisturizers, personal cleansing products, occlusive drug delivery patches, powders, wipes, hair conditioners, hair tonics, shampoos, hair colorants, skin treatment emulsions, shaving creams, antiperspirants, deodorants, and the like.

“Regulating hair growth,” namely mammalian hair growth, includes reducing, modulating, inhibiting, attenuating, retarding, and/or the diminution of hair growth.

“Reduction/Inhibition of hair growth,” as referenced herein, is demonstrated when the frequency of hair removal is reduced, or the appearance and/or feel of mammalian hair is improved wherein the subject perceives less hair on the treated site (i.e., hair is perceived to be softer, finer, less noticeable), or quantitatively, when the weight of the hair removed by shaving (i.e., hair mass) is reduced thereby improving the ease, frequency, and effectiveness of shaving of a mammal.

“Mammalian hair,” as referenced herein, includes hair on any part of the body of a mammal and may include facial, cranial, or body hair. Male facial hair commonly refers to the beard, moustache, eyebrows and sideburns hair, but may include any area of the face and/or neck. Female facial hair (predominantly vellus, but can include terminal hair) commonly refers to eyebrows, upper lip, chin, and cheeks area, but may also include any area of the face and/or neck. Other areas of hair growth typically desired to be regulated include underarms, bikini area, legs, arms, back, and chest.

The phrases “improving the appearance and/or feel” and “the appearance and/or feel of mammalian hair is improved,” as used herein, refer to noticeable improvement in the appearance and/or feel of the hair on the skin such that it is perceived to be softer, finer, less noticeable. Additionally, the ease, frequency, and effectiveness of shaving will be perceived by the mammal. Reduction of hair growth is demonstrated when the frequency of hair removal is reduced, or the subject perceives less hair on the treated site, or quantitatively, when the weight of hair removed by shaving (i.e., hair mass) is reduced.

The term “keratinous tissue,” as used herein, refers to keratin-containing layers disposed as the outermost protective covering of mammals (e.g., humans, dogs, cats, etc.) which includes, but is not limited to, skin, hair, etc.

The term “topical application,” as used herein, means to apply or spread the compositions of the present invention onto the surface of the keratinous tissue.

The term “dermatologically-acceptable,” as used herein, means that the compositions or components thereof so described are suitable for use in contact with mammalian keratinous tissue without undue toxicity, incompatibility, instability, allergic response, and the like.

The term “safe and effective amount,” as used herein, means an amount of a compound or composition sufficient to significantly induce a positive benefit, preferably a hair growth regulating benefit, or positive hair appearance or feel benefit, including independently or in combinations, the benefits disclosed herein, but low enough to avoid serious side effects, i.e., to provide a reasonable benefit to risk ratio, within the scope of sound judgment of the skilled artisan.

The term “ambient conditions,” as used herein, refers to surrounding conditions under about one atmosphere of pressure, at about 50% relative humidity, and at about 25° C., unless otherwise specified.

The phrase “treating an/the expanse of skin with an acute hair growth technology,” as used herein, includes shaving, epilating, contacting the skin with a depilatory, waxing, and the like.

### I. Nitrone Derivative

Personal care compositions of the present invention comprise a safe and effective amount of at least one nitrone derivative. Nitrones are capable of irreversibly capturing electrons and/or free radicals, thereby reducing the relative amount of oxidative potential in a microenvironment. Thus, these materials have been referred to as spin traps since the ability to detect a free radical via spectroscopic means involves monitoring the spin resonance of free radicals. By irreversibly binding the free radical, the spectroscopic signal becomes reduced due to the free radical becoming trapped by a nitrone.

A representative, non-limiting list of nitrone derivatives (or “spin traps”) includes α-phenyl butyl nitrone (PBN), PBN doxylcyclohexane radicals, 5,5-dimethyl pyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, or spin-trapping derivatives thereof, or mixtures thereof.

Without being limited by theory, it is believed that the compositions of the present invention containing free radical spin traps are able to modulate hair growth by impacting several key processes. These include reducing mRNA expression levels, and thereby protein levels, of inducible nitric oxide synthase (iNOS), matrix metalloproteases (MMPs), and cyclooxygenase-2 (COX-2). Additionally, spin traps have been found to inhibit iNOS and COX-2 enzymatic activity directly. Since nitric oxide (NO) plays a key signal transduction role in stimulating follicular cell growth, as well as provide a signal for stimulating and maintaining angiogenesis, it is hypothesized that reduction of iNOS enzyme levels will reduce the amount of NO being produced in follicular cells. This in turn can reduce hair growth rates due to a lower vasculature system that is essential for anagen state of hair follicles. MMPs are key components in restructuring of the extracellular matrix during follicular progression through the dermis of skin in early anagen. Additionally, MMPs play a role in angiogenesis, a key process for vascularization of the hair follicle during early anagen, as well as maintenance of the vasculature bed during all of anagen. Thus, inhibition of MMPs and reduced levels can in turn lead to a reduction in hair growth rates. Finally, since prostaglandins play a critical role in regulating cellular proliferation, inhibition of COX-2 can lead to a reduced proliferative index of hair follicles cells, ultimately leading to decreased hair growth rates.

The nitrone derivative is included at a level of from about 0.01% to about 10%, preferably from about 0.5% to about 7%, and more preferably from about 1% to about 5%, by weight of the composition. Other inclusion levels are also contemplated by the present invention.

### II. Dermatologically Acceptable Carrier

Compositions of the present invention also comprise a dermatologically acceptable carrier. The carrier is preferably present in an amount from about 50% to about 99.99%, preferably from about 60% to about 99.9%, more preferably from about 70% to about 98%, and even more preferably from about 80% to about 95%, by weight of the composition.

The carrier can be in a wide variety of forms. For example, emulsion carriers, including, but not limited to, oil-in-water, water-in-oil, silicone-in-water, water-in-silicone, water-in-oil-in-water, and oil-in-water-in-silicone emulsions, are useful herein.

Preferred carriers comprise an emulsion such as oil-in-water emulsions and water-in-oil emulsions, e.g., silicone-in-water or water-in-silicone emulsions. As will be understood by the skilled artisan, a given component will distribute primarily into either the water or oil phase, depending on the water solubility/dispensability of the component in the composition. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317 324 (1986).

Suitable emulsions may have a wide range of viscosities, depending on the desired product form. Exemplary low viscosity emulsions, which are preferred, have a viscosity of about 50 centistokes or less, more preferably about 10 centistokes or less, even more preferably about 5 centistokes or less.

Compositions of the present invention can also comprise other known topical carriers, and can also comprise oral carriers.

### III. Optional Ingredients

Compositions of the present invention may contain a wide variety of other ingredients. The optional ingredients, when incorporated into the composition, should be suitable for use in contact with human keratinous tissue without undue toxicity, incompatibility, instability, allergic response, and the like within the scope of sound judgment. When incorporated, the optional ingredients are present in the composition at levels of from about 0.01% to about 20%, more preferably from about 0.05% to about 10%, still more preferably from about 0.1% to about 5%, by weight of the composition.

It is to be understood that headings and classifications herein are made for the sake of convenience and are not intended to limit an ingredient to a particular application or applications listed, particularly with respect to interpreting the appended claims.

A. Hair Growth Inhibiting Compounds

The compositions of the present invention may also comprise other hair growth inhibiting compounds besides the nitrone derivative. Preferred additional hair growth inhibiting compounds include hexamidine, green tea catechins, soy proteins (preferably hydrolyzed proteins), butylated hydroxytoluene, agmatine, aminoguanidine, and mixtures thereof. A representative, non-limiting list of other suitable hair growth inhibiting compounds includes the following classes and examples.

1. Natural Plant Extracts

Natural plant extracts useful herein include, but are not limited to, compounds extracted from any part of the plant of saw palmetto, willow herb, pumpkin seed, creosote, sea-buckthorn oil, capsicum, Echinacea angustifolia, Echinacea purpurea, Lithosperumum, Rosaceae, Sanguisorba officinalis, Tropaeolum majus, white birch and rubiaceae plant groups, Juniperus genus, malt, from genus Centipeda, Cinnamonum verum, Curcurbita pepo, Epilobium roseum, Salvia officinalis, Cassia obtusifoila Linne, Pleione genus, Curcuma longa, Salix alba, Hamamelis virginiana, Diopyros kaki, Hydrangea macrophylla, Hydrangea serrata, Iridaceae genus, Moraceae Humulus, Ikurinin, Regulo plant (Abelmoschus moschatus), Wolo plant (Borassus flabellifer), Hedera helix, Lithospermu, Scutellaria genus, tomato, Commiphora myrrha, Cymbopogon nardus, Lagerstroemia speciosa, Phyllanthus nuriri, Smilax zeylanica, Woodfordia fruticosa, Cistanche salsa, Larrea divaricata, Plantago asiatica, Stachys sieboldii, lavender, lemon, carrot, ginger, clove, honey, juniper, almond, palmarosa, eucalyptus, rosemary, sugars, Coix lachryma-jobi, bur marigold infusion, bacterium ribosomes, conifer extract, daisy infusion, tea tree oil, spearmint, honey, ant eggs, Bowman Birk inhibitor, peach oil, essential oils, aloe, elasatin decomposition enzyme inhibitor, peptides, plant fruit enzymes, shogaol, zingerone, zingiberol, and zingiberone

2. Metabolic Modulators

Metabolic modulators useful herein include, but are not limited to, 5′-p-fluorosulphonyl benzoyl adenosine, 5-keto-D-fructose, 5-keto-D-fructose-1,6-bisphosphate, 6-amino-6-deoxy-glucose, inhibitor of a cysteine pathway enzyme, guanidino succinic acid, cysteine sulphinic acid, phosphoglycerate, cysteamine, cysteine sulphinic acid, cysteinyl-glycine, D-cysteine, inhibitor of a cholesterol pathway enzyme, inhibitor of the formation of glycoproteins, N-acetylcysteine (NAC), D-mannosamine, N-alpha-(p-tosyl)-L-lysine chloromethyl ketone, N-acetyl-beta-D-mannosamine, oxaloacetic acid, finasteride, arginase inhibitor, N-phosphonoacetyl-aspartic acid, N-alpha-acetyl-L-arginine, N-alpha-benzoyl-L-argininamide, N-alpha-benzoyl-L-arginine, N-alpha-benzoyl-L-arginine methyl ester, NG-L-arginine benzyl ester, NG-nitro-L-arginine, NG-nitro-L-arginine methyl ester, dithiothreitol, glutathione, homocysteine, lipoic acid, 2-mercaptoethanol, mecaptopropionic acid, thiodiglycol, thiodiglycolic acid, thioglycerol, thioglycolic acid, thiolactic acid, thiomalic acid, thiopropionic acid, thiosalicylic acid, thioxanthine, H-homoarginine, L-alanosine, L-argininamide, L-asparaginamide, L-cysteine methyl ester, alpha-methyl-DL-methionine, dimethyl cysteamine, sulfotransferase inhibitors, N(G)-methyl-L-arginine, alpha.-fluoromethylhistidine, inhibitors of glutamine metabolism, glutathione synthesis stimulators, fatty acids, chelating agents, pravastatin, rivastatin, simvastatin, squalestatin, fluvastatin, mevastatin, mevinolin, lovastatin, cysteine, chlorotaurine, 2-mercaptopropionic acid, diethyldithiocarbamic acid, aromatase inhibitors, glutathione S-transferase modulators, peptide or trisamine carrying fatty acid ester and dithioalkanoyl groups, sorbic acid, vitamin K, vitamin F, and phloretin.

3. Anti-Proliferatives

A representative, non-limiting list of anti-proliferatives useful herein include difluoromethylornithine (DFMO), polyamine transport inhibitors, antizyme modulators, methacycline, protein kinase C inhibitors, protein-tyrosine kinase inhibitors, tyrphostins and tryphostins, cyclooxygenase inhibitors, 5-alpha-reductase inhibitors, adenylsuccinate synthetase inhibitor, aspartate transcarbamylase inhibitor, gammaglutamyl transpeptidase inhibitor, ornithine decarboxylase inhibitors, non-steroidal anti-inflammatory drugs (NSAIDS), lipoxygenase inhibitors and stimulants, nordihydroguaianetic acid (NDGA), inhibitor of alkaline phosphatase, doxycycline, minocycline, taxodione, taxodone, bacteriostatic or haemostyptic agent, especially stannous fluoride, alpha-ethyl-ornithine, nalidixic acid, tetracycline, inhibitors of the hypusine biosynthetic pathway, methacycline, methylglyoxal bis(guanylhydrazone), bromocryptine, trihydroxypurine, etoposide, guanidino succinic acid, matlystatin-B, 5′-deoxy-5′-(N-methyl-N-(2-aminooxy-ethyl)-aminoadenosine (MAOEA), 5′-deoxy-5′-methyl-thioadenosine, doxycycline, pyrimidine-cyanoguanidine derivatives, substituted amidine or guanidine, butyric acid derivatives, hydroxamic acid and analogues, medroxyprogesterone acetate, megestrol acetate, melengestrol acetate, nomegestrol acetate, mycophenolic acid, cyanoguanidine derivatives, and diethyl glyoxal bis(quanylhydrazone).

4. Signal Transduction Modulators

Signal transduction modfulators useful herein include, but are not limited to, Br-cAMP, E6AP-binding polypeptides, ethoxyquin, anti-angiogenic steroids, CDK binding proteins, chimeric polypeptide with cyclin-dependent kinase (CDK) binding motif, suppressor of angiogenesis, alpha- or gamma-linolenic acid, EGF and analogues, Hairless protein and analogues, estrogen agonists or antagonists, proteoglycans or glycosaminoglycans, phytoestrogen, hedgehog antagonists, patched antagonists, interleukin-1 antagonist, alpha-TNF antagonist, leuteinizing hormone-releasing hormone and analogues, GnRH inhibitors, Heptapeptide luteinizing hormone releasing hormone (LHRH) analogs, 1-halomethyl-5alpha-androstanes and delta-androstenes, 3-oxo-4-aza-5 alpha-androstane derivatives, finasteride, spironolactone, propyl gallate, eicosapentaenoic acid, lavendustin A, activin A, androgen receptor blockers, quercetin, protocatechuic acid and aldehyde, methyl caffeate, apigenin, caffeic acid, progestins and antiprogestins, vitamin D and analogues including previtamin D and provitamin D, androstenedione analogues, lipoxydase, spironolactone, cyproterone acetate, progesterone, and melatonin.

5. Proteases and Protease Inhibitors

Protease and protease inhibitors useful herein include, but are not limited to, 1,10-phenanthroline, elastase inhibitors, caspase inhibitors, cathepsin inhibitors, papain, inhibitors of trypsin and analogues, chymotrypsin, pepsin, bromelain, ficin, pancreatin, and marimistat.

6. Other Hair Growth Inhibiting Compounds

Other hair growth inhibiting compounds useful herein include phlondrin, agaric acid, vernolepin, D-penicillamine, ethacrynic acid, eupacunin, euparotin acetate, diethylaminomalonate, protocatechuic aldehyde, non-elastomeric polyolefin resin, partially fluorinated polyolefin resin, quinaldic acid, 1,8-diaminooctane, 2-methyl-6-heptyne-2,5-diamine, 3-carboxypropyl disulphide, 5-(N-benzyloxycarbonyl)-1-phenylalanamidomethyl)-3-bromo-4,5-dihydroisoxazole, 6-heptyne-2,4-diamine, actinonin, batimistat, captopril, diethyl aminomalonate, diethyldithiocarbamic acid, estramustine, ethacrynic acid, meso-dimercaptosuccinic acid, N-[N[((R)-1-phosphonopropyl)-(S)-leucyl]-(S)-phenylalanine-N-methylamide, N-phosphonalkyl dipeptides, oxaloacetic acid, phosphocysteamine, S-carbamyl-L-cysteine, S-trityl-L-cysteine, sulphasalazine, thiosalicylic acid, tyramine, 2-difluoromethyl-, 2,5-diamino pentanoic acid, herbimycin, HNMPA (AM)3, O-p-nitrohydroxylamine, cromoglycate, quinoline-3-carboxamide, 16 alpha- or beta-substituted 4-aza-5 alpha-androst-1-en-3-ones, 2-aryl-indole derivatives, 2-phenyl-3-aminoalkyl-indole derivatives, 5 alpha-androstan-3-ones, 5-(aminocarbonylalkyl)-3-(heterobicyclyl-alkylaminoalkyl)-2-phenylindole derivatives, 6-azaindole derivatives, 7-azaindole derivatives, aryl-imidazo-pyridines, carboxyalkylamine derivatives, malonamide derivatives, 2-indole carboxylic acid derivatives, aminopropanes, diethylenediamines, histamine antagonist, phenothiazines, tetrazolyl-benzofuran carboxamides, tetrazolyl-benzothiophene carboxamides, 17alpha-hydroxy-4,9(11)-pregnadiene-3,20-dione derivatives, benzothiophene derivatives, (−)cis 6(S)-phenyl-5(R)-[4-(2-pyrrolidin-1-ylethoxyphenyl]-5,6,7,8-tetrahydronaphthelen-2-ol D-tartrate (I), tetrahydronaphthalene derivatives, tetrahydroisoquinolines, tetrahydroisoquinoline derivatives, tetrahydroisoquinoline derivatives, 3-(anilinomethylene)oxindole derivatives, benzo-[f]-quinolin-3-one derivative, ((S-(−)-N-(alpha-ethylbenzyl)-3-hydroxy-2-phenylquinoline-4-carboxamide), 24-ethyl-(delta)4,22-cholestadien-3-one, benzoic acid lactone ether, copper, iron, zinc, 1 dehydromelengestrol acetate, 1-dehydromegestrol acetate, chlormadinone acetate, cyproterone acetate, hydrindanes, diazo compounds, tetrahydroisoquinolines, tetrahydronaphthalenes, 3-amino-2,3-dihydro benzoic acid, 6-fluoro-2,5-diamino hexanoic acid, (S)-2-amino-4-amino oxy-butyric acid, triarylmethane compounds, perfluoro-substituted aniline derivatives, 17alpha-propyltestosterone, 4-androstene-3-one 17beta-carboxylic acid, (4R)-5,10-seco-19-norpregna 4,5-diene-3,10,20-trione, chlormadinone acetate, 2-substituted 6-tetra hydronaphthyl or indanyl naphthalene derives, 2-phenyl-benzothiophene derivatives, 2-arylimino-oxaza or thiaza heterocyclic compounds, indole derivatives, dormant cell extracts, N-substituted benzyl- or thienylmethyl-4-pyridone compounds, (1H)-benzo(c)quinolizin-3-one derivatives, citric acid, Dead Sea salts, visaborol, chlorophenol, o-phenylphenol, phenol, niphtolide, 2-amino-5-substituted benzophenone, aniline derivatives, camphor oil, citric acid, conjugate comprising active agent substituted with amino acid, 11-beta-aryl-17-spiro-pyrrolin-2-ylidene N-oxide steroid, phenyl imidazolidines, coumarin derivatives, borneol, cineole, linalool, methyl heptenone, thiomolybdate compound, and trifluoroanilide derivatives.

Polyamine transport inhibitors and antizyme modulators may also be incorporated into the compositions of the present invention, such as those, for example, disclosed in U.S. Patent Application Nos. 2004/0209926, 2005/0176828, and 2005/0245615.

B. Depilatories

Compositions of the present invention may optionally contain a depilatory. As used herein, “depilatory” means an agent capable of removing hair from the skin by cleaving the disulfide bonds in hair keratin, thereby causing the hair fiber to disintegrate. Preferred depilatories useful in the subject invention include ammonium thioglycolate, barium sulfate, calcium thioglycolate, ethanolamine thioglycolate, potassium thioglycolate, sodium thioglycolate, thioglycolic acid and thioacetic acid. Examples of suitable depilatories are described in further detail in U.S. Pat. No. 5,897,857.

C. Desquamation Actives

A safe and effective amount of a desquamation active may be added to the compositions of the present invention. One desquamation system that is suitable for use herein comprises sulfhydryl compounds, salicylic acid, and zwitterionic surfactants.

D. Particulate Materials

The compositions of the present invention may comprise one or more particulate materials. Nonlimiting examples of particulate materials useful in the present invention include particles, pigments, and cross-linked silicone elastomers.

Particulate materials useful herein include, but are not limited to, colored and uncolored pigments, interference pigments, inorganic powders, organic powders, composite powders, optical brightener particles, exfoliants and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. These particulate materials may provide a wide range of functions, including but not limited to modifying skin feel, masking the appearance of certain skin characteristics such as blotchy areas, age spots, freckles, fine lines, wrinkles, and pores, absorbing excess skin sebum/oils, reducing skin shine, improving application properties of the composition, masking the color of other components of the composition, filling in skin pores, lines and wrinkles, and reducing migration of liquid materials on the skin. There are no specific limitations as to the pigment, colorant or filler powders used in the composition.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, sericite, aluminum oxide, silicone resin, barium sulfate, calcium carbonate, cellulose acetate, PTFE, polymethyl methacrylate, starch, modified starches such as aluminum starch octenyl succinate, silk, glass, fibers, ground seeds, pumice, and mixtures thereof. Especially preferred are spherical powders with an average primary particle size from about 0.1 to about 75 microns, preferably from about 0.2 to about 30 microns.

Particulate materials include silicone elastomers and compositions made from the same. Suitable organopolysiloxane gel compositions are dimethicone/vinyl dimethicone crosspolymers swollen in an appropriate solvent. Such dimethicone/vinyl dimethicone crosspolymers are supplied by a variety of suppliers including Dow Corning (DC 9040™ and DC 9041™), General Electric (SFE 839™), Shin Etsu (KSG-15™, KSG-16™, KSG-18™ [dimethicone/phenyl vinyl dimethicone crosspolymer]) and lauryl dimethicone/vinyl dimethicone crosspolymers supplied by Shin Etsu (e.g., KSG-31™, KSG-32™, KSG-41™, KSG-42™, KSG-43™, and KSG-44™). Alternatively, organopolysiloxane elastomer powders can be used, suitable examples include vinyl dimethicone/methicone silesquioxane crosspolymers like Shin-Etsu's KSP-100™, KSP-101™, KSP-102™, KSP-103™, KSP-104™, KSP-105™, hybrid silicone powders that contain a fluoroalkyl group like Shin-Etsu's KSP-200™, and hybrid silicone powders that contain a phenyl group such as Shin-Etsu's KSP-300™; and Dow Corning's DC 9506™.

Also useful herein are interference pigments. Interference pigments, for purposes of the present invention are defined as thin platelike layered particles having two or more layers of controlled thickness with different refractive indices that yield a characteristic reflected color from the interference of typically two, but occasionally more, light reflections, from different layers of the platelike particle. Examples of interference pigments are micas layered with about 50-300 nm films of TiO2, Fe2O3, silica, tin oxide, and/or Cr2O3. Such pigments are often pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example, Rona (Timiron™ and Dichrona™), Eckart (e.g. Prestige and Prestige Silk lines). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX TiO2 series, SAT-T CR837, a rutile TiO2). Particularly preferred are charged dispersions of titanium dioxide, as are disclosed in U.S. Pat. No. 5,997,887.

The pigments/powders useful herein can be surface treated to provide added stability of color and/or for ease of formulation. Non-limiting examples of suitable coating materials include silicones, lecithin, amino acids, metal soaps, polyethylene and collagen. These surface treatments may be hydrophobic or hydrophilic, with hydrophobically treatments being preferred. Particularly useful hydrophobic pigment treatments include polysiloxane treatments such as those disclosed in U.S. Pat. No. 5,143,722.

E. Panthenol and Pantothenic Acid Derivatives

Panthenol and its derivatives include, but are not limited to, D-panthenol ([R]-2,4-dihydroxy-N-[3-hydroxypropyl)]-3,3-dimethylbutamide), DL-panthenol, pantothenic acids and their salts, preferably the calcium salt, panthenyl triacetate, royal jelly, panthetine, pantotheine, panthenyl ethyl ether, pangamic acid, pantoyl lactose, Vitamin B complex, or mixtures thereof.

The term “pantothenic acid derivative,” as used herein, refers to those materials that conform to the formula:

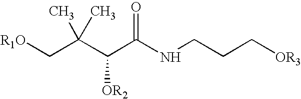

wherein R₁, R₂ and R₃ are hydrogen, C2-C20 hydrocarbons, C2-C20 carboxylic acid esters, or combinations thereof, provided that not more than two of R₁, R₂ and R₃ are hydrogen. Preferably, R₁, R₂ and R₃ are independently selected from hydrogen, C2-C8 hydrocarbons, C2-C8 carboxylic acid esters, or combinations thereof, more preferably, R₁ and R₂ are hydrogen, and R₃ is a C2-C8 hydrocarbon, C2-C8 carboxylic acid ester, or combinations thereof; even more preferably, R₁ and R₂ are hydrogen and R₃ is ethyl. The pantothenic acid derivatives may be derived or otherwise obtained from any known source, which may include pantothenic acid or materials other than pantothenic acid, so long as the resulting material has the above defined chemical formula.

Specific non-limiting examples of pantothenic acid derivatives for use herein include ethyl panthenol, panthenyl triacetate, and combinations thereof. Preferred are the d-isomeric forms of such derivative forms, most preferably d-ethyl panthenol.

F. Tanning Actives

Compositions of the present invention may comprise a self-tanning agent. As used herein, the term “self-tanning agent” includes α-hydroxy aldehydes and ketones such as dihydroxyacetone and structurally related compounds, and all such agents that are similarly useful in producing or inducing the artificial tanning process in human skin.

α-hydroxy aldehydes or ketones useful in compositions of the present invention include those in accordance with the following formula:

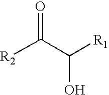

wherein R₁ is H, CH₂OH, CHOHCH₂OH, CH(OH)CH(═O), CH(OCH₃)CH(═O), CH(NH₂)CH(═O), or CH(NH-Phenyl)CH(═O); and R₂ is H or CH₂OH.

Dihydroxyacetone (DHA) itself may be represented by the following general structural formula:

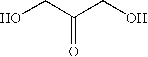

Other tanning compounds useful herein include glyceraldehyde, 2,3-dihydroxy-succindialdehyde, 2,3-dimethoxysuccindialdehyde, erythrulose, erythrose, 2-amino-3-hydroxy-succindialdehyde, 2-benzylamino-3-hydroxy-succindialdehyde.

G. Anti-Acne Actives

The compositions of the present invention may comprise a safe and effective amount of one or more anti-acne actives. Examples of useful anti-acne actives include resorcinol, sulfur, salicylic acid, erythromycin, zinc, etc.

H. Anti-Wrinkle Actives/Anti-Atrophy Actives

Exemplary anti-wrinkle/anti-atrophy actives suitable for use in compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., salicylic acid, glycolic acid), keto acids (e.g., pyruvic acid), ascorbic acid (vitamin C), phytic acid, lipoic acid; lysophosphatidic acid, skin peel agents (e.g., phenol and the like), retinoids such as retinol and retinyl propionate, flavonoids (e.g., flavanones, chalcones, isoflavones, flavones, etc.), boswellic acid, stilbenes, cinnamates, resveratrol, kinetin, zeatin, dimethylaminoethanol, peptides from natural sources (e.g., soy peptides), salts of sugar acids (e.g., Mn gluconate), Coenzyme Q₁₀ (ubiquinone, vitamin Q₁₀), terpene alcohols (e.g., farnesol), peptides, vitamin B3 and other vitamin B compounds (e.g., thiamine (vitamin B1), pantothenic acid (vitamin B5), carnitine (vitamin Bt), riboflavin (vitamin B2), cobalamine (vitamin B12), pangamic acid or diisopropylamine dichloroacetate (vitamin B15's), and their derivatives and salts (e.g., HCl salts or calcium salts)).

I. Anti-Oxidants/Radical Scavengers

The compositions of the present invention may include a safe and effective amount of an anti-oxidant/radical scavenger. The anti-oxidant/radical scavenger is especially useful for providing protection against UV radiation that can cause increased scaling or texture changes in the stratum corneum and against other environmental agents, which can cause skin damage.

Anti-oxidants/radical scavengers such as ascorbic acid (vitamin C) and its salts, ascorbyl esters of fatty acids, ascorbic acid derivatives (e.g., magnesium ascorbyl phosphate, ascorbyl glucoside), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox^{R}), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol sorbate.

J. Chelators

The compositions of the present invention may comprise a safe and effective amount of a chelator or chelating agent. As used herein, “chelator” or “chelating agent” means an active agent capable of removing a metal ion from a system by forming a complex so that the metal ion cannot readily participate in or catalyze chemical reactions.

K. Anti-Inflammatory Agents

Anti-inflammatory agents may enhance skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency. Anti-inflammatories can be selected from several classes. One is comprised of steroidal anti-inflammatory agents, including but not limited to, corticosteroids. The preferred steroidal anti-inflammatory for use is hydrocortisone.

A second class of anti-inflammatory agents, which is useful in the compositions, includes the nonsteroidal anti-inflammatory agents. Such compounds are known in the art as non-steroidal anti-inflammatory agents (“NSAIDS”) and are described in detail, along with methods for manufacture in the following U.S. Pat. Nos. 5,280,045; 4,708,966; 5,189,066; 5,510,361; 5,189,066; 5,476,876; and 5,684,204.

Mixtures of these non-steroidal anti-inflammatory agents may also be employed, as well as the dermatologically acceptable salts and esters of these agents.

Finally, so-called “natural” anti-inflammatory agents are useful in compositions of the present invention. Such agents may suitably be obtained as an extract by suitable physical and/or chemical isolation from natural sources (e.g., plants, fungi, and by-products of microorganisms).

Additional anti-inflammatory agents useful herein include allantoin and compounds of the Licorice (the plant genus/species Glycyrrhiza glabra) family, including glycyrrhetic acid, glycyrrhizic acid, and derivatives (e.g., salts and esters). Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred. The active component of these anti-inflammatory agents (e.g., bisabolol, glycyrrhetinate esters) may also be obtained via extraction from natural sources or prepared synthetically.

M. Anti-Cellulite Agents

The compositions of the present invention may comprise a safe and effective amount of an anti-cellulite agent. Suitable agents may include, but are not limited to, xanthine compounds (e.g., caffeine, theophylline, theobromine, and aminophylline).

N. Topical Anesthetics

The compositions of the present invention may also comprise a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

0. Skin Lightening Agents

The compositions of the present invention may comprise a skin lightening agent. Suitable skin lightening agents include those known in the art, including kojic acid, arbutin, tranexamic acid, ascorbic acid and derivatives, e.g., magnesium ascorbyl phosphate or sodium ascorbyl phosphate or other salts of ascorbyl phosphate.

P. Antimicrobial and Antifungal Actives

The compositions of the present invention may comprise an antimicrobial or antifungal active. Such actives are capable of destroying microbes, preventing the development of microbes or preventing the pathogenic action of microbes.

Q. Sunscreen Actives

Sunscreen actices useful in the compositions include those disclosed in U.S. Pat. Nos. 4,937,370 and 4,999,186. The sunscreen agents disclosed therein have, in a single molecule, two distinct chromophore moieties, which exhibit different ultra-violet radiation absorption spectra. One of the chromophore moieties absorbs predominantly in the UVB radiation range and the other absorbs strongly in the UVA radiation range.

R. Conditioning Agents

The compositions of the present invention may comprise a conditioning agent selected from the group consisting of humectants, moisturizers, or skin conditioners. A variety of these materials can be employed and include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy compounds such as sorbitol, mannitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose, sucrose, etc.); hyaluronic acid; lactamide monoethanolamine; acetamide monoethanolamine; and mixtures thereof. Also useful herein are the propoxylated glycerols and various C₁-C₃₀ monoesters and polyesters of sugars and related materials.

Preferably, the conditioning agent is selected from the group consisting of glycerol, urea, guanidine, sucrose polyester, and combinations thereof.

S. Alkane Polyols

Compositions of the present invention may comprise an alkane polyol. A representative, non-limiting list of alkane polyols includes 1,2-hexanediol; 2-ethyl-1,3-hexanediol; 1,2-heptanediol; 1,7-heptanediol; 1,2-octanediol; 1,9-octanediol; and 1,9-nonanediol. Hexanediols are preferred alkane polyols. Alkane polyols are particularly useful in composition embodiments including hexamidine.

T. Thickening Agent

The compositions of the present invention can comprise one or more thickening agents.

1. Carboxylic Acid Polymers

The compositions of the present invention can optionally comprise carboxylic acid polymers. These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. Examples of carboxylic acid polymer thickeners useful herein are those selected from the group consisting of carbomers, acrylates/C₁₀-C₃₀ alkyl acrylate crosspolymers, and mixtures thereof.

2. Crosslinked Polyacrylate Polymers

The compositions of the present invention can optionally comprise crosslinked polyacrylate polymers useful as thickeners or gelling agents including anionic, cationic and nonionic polymers, with the cationics being generally preferred.

3. Polyacrylamide Polymers

The compositions of the present invention can optionally comprise polyacrylamide polymers, especially nonionic polyacrylamide polymers including substituted branched or unbranched polymers. Preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

4. Polysaccharides

A wide variety of polysaccharides are useful herein. “Polysaccharides” refer to gelling agents that contain a backbone of repeating sugar (i.e., carbohydrate) units. Nonlimiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage.

Other useful polysaccharides include scleroglucans comprising a linear chain of (1-3) linked glucose units with a (1-6) linked glucose every three units, a commercially available example of which is Clearogel™ CS11 from Michel Mercier Products Inc. (Mountainside, N.J.).

5. Gums

Other thickening and gelling agents useful herein include materials that are primarily derived from natural sources. Nonlimiting examples of these gelling agent gums include materials selected from the group consisting of acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

6. Polyquaternium-37

The compositions of the present invention may also comprise the synthetic cationic polymer Polyquaternium-37 (methacryloylethyl trimethyl ammonium chloride homopolymer). This polymer may be added to the composition as a powder or as a liquid dispersion. This polymer is commercially available under the tradenames Synthalen (3V Sigma), Ultragel 300 (Cosmetic Rheologies Ltd), Rheocare CTH(E) (Cosmetic Rheologies Ltd.), Salcare SC95 and Salcare SC96(Ciba Specialty Chemicals). Polyquaternium-37 is particularly useful in composition embodiments including hexamidine.

### Method of Making

The compositions of the present invention are generally prepared by conventional methods such as are known in the art of making personal care compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), and use of appropriate photostability approaches (e.g., incorporation of sunscreen/sunblock, use of opaque packaging).

### Methods of Use

The compositions of the present invention are useful for regulating keratinous tissue, particularly hair growth and mammalian skin condition. Such regulation of keratinous tissue conditions can include prophylactic and therapeutic regulation. It may also include providing a more noticeable improvement, both tactile and visual, in the appearance and feel of hair on the skin of a mammal. Such methods may provide ease, frequency, and effectiveness of shaving on a mammal, as slower re-growth of the hair allows treated skin to be shaved less frequently, thereby reducing irritation and erythema, and wounding events such as nicks and/or cuts. By slowing down the re-growth of the hair, the hair can become less noticeable, softer, and/or finer and the skin left feeling smoother and/or silkier. Additional benefits may include improvements in the ease of shaving and increased shaving efficiency. Thus, the compositions of the present invention can be useful in inhibiting hair growth, reducing shaving frequency, improving ease of shaving, decreasing shaving frequency, making hair softer and/or finer, making hair less noticeable, slowing the re-growth of hair, reducing erythema and/or irritation to skin, making skin smoother and/or silkier, and/or improving the hair removal process.

Compositions of the present invention may also be useful regulating skin conditions, including, but are not limited to, thickening keratinous tissue (i.e., building the epidermis and/or dermis layers of the skin and where applicable the keratinous layers of the nail and hair shaft), preventing and/or retarding atrophy of mammalian skin, preventing and/or retarding the appearance of spider vessels and/or red blotchiness on mammalian skin, treating (i.e., preventing and/or retarding the appearance of) dark circles under the eye of a mammal, preventing and/or retarding sallowness of mammalian skin, regulating (i.e., preventing and/or retarding) sagging of mammalian skin, softening and/or smoothing lips, hair and nails of a mammal, preventing and/or relieving itch of mammalian skin, regulating skin texture (e.g., wrinkles and fine lines), regulating the appearance of shiny skin, treating (i.e., preventing and/or retarding the appearance of) cellulite, increasing the rate of skin turnover, and improving skin color (e.g., redness, freckles).

Regulating keratinous tissue condition is preferably practiced by applying a composition in the form of a skin lotion, cream, gel, foam, ointment, paste, serum, stick, emulsion, spray, conditioner, tonic, antiperspirant, deodorant, cosmetic, lipstick, foundation, nail polish, after-shave, or the like which is preferably intended to be left on the keratin structure for some esthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, still more preferably for at least several hours, e.g., up to about 12 hours. Any part of the external portion of the face, hair, and/or nails can be treated; e.g., face, lips, under-eye area, upper lip, eyelids, scalp, neck, torso, arms, underarms, hands, legs, feet, fingernails, toenails, scalp hair, eyelashes, eyebrows, etc. The composition can be applied with the fingers or with an implement or device (e.g., pad, cotton ball, applicator pen, spray applicator, and the like).

Another approach to ensure a continuous exposure of compositions of the present invention is by use of a patch. Such an approach is particularly useful for problem skin areas needing more intensive treatment (e.g., facial crows feet area, frown lines, under eye area, upper lip and the like). The patch can be occlusive, semi-occlusive or non-occlusive and can be adhesive or non-adhesive. The composition can be contained within the patch or be applied to the skin prior to application of the patch. The patch can also include additional actives such as chemical initiators for exothermic reactions such as those described in U.S. Pat. Nos. 5,821,250; 5,981,547; and 5,972,957 to Wu, et al. The patch is preferably left on the skin for a period of at least about 5 minutes, more preferably at least about 15 minutes, more preferably still at least about 30 minutes, even more preferably at least about 1 hour, still more preferably at night as a form of night therapy.

In preferred embodiments, the composition is chronically applied to the skin. By “chronic topical application” is meant continued topical application of the composition over an extended period during the subject's lifetime, preferably for a period of at least about one week; more preferably for a period of at least about four, six or eight weeks; for at least about three to six months; or for at least about one year. Typically applications would be on the order of about once or twice per day over such extended periods, however application rates can vary from about once per week up to about three times per day or more.

In one preferred embodiment, the compositions are applied for at least one complete hair growth cycle. An expanse of tissue containing hair follicles will generally contain follicles at different phases of the hair growth cycle; that is, the individual hair follicles are asynchronous with respect to which hair growth cycle phase they are in. Thus, in order for all, or substantially all, of the hair follicles within a treatment area to be exposed to the compositions of the present invention, it is preferred to apply the composition for at least one complete hair growth cycle. It is known that hair growth cycles vary depending on the bodily location and the type of hair being regulated. Thus, the chronic application timeframe will likewise vary.

A wide range of quantities of the compositions of the present invention can be employed to provide an appearance and/or feel benefit. Quantities of the present compositions, which are typically applied per application, are, in mg composition/cm² skin, from about 0.1 mg/cm² to about 20 mg/cm². A particularly useful application amount is about 0.5 mg/cm² to about 10 mg/cm².

Hair growth management regimens employing personal care compositions, as described above, are also provided by the present invention. In one exemplary embodiment, acute hair growth technologies are used in combination with topical application of the personal care compositions disclosed herein. Acute hair growth technologies and activities include depilatories; razors, razor blades, and shaving; waxes and waxing; and repilators, for example. Acute hair growth technologies also include energy delivery devices. The energy delivery device may deliver energy in a variety of forms, including but not limited to energy in the form of light, heat, sound (including ultrasonic waves), magnetic energy, electromagnetic energy (including radiofrequency waves and microwaves), and combinations thereof. Hair growth management regimens include employing the acute hair growth technologies/activities at a first frequency and topical application of the personal care compositions at a second frequency, wherein the second frequency is preferably greater than the first frequency. For example, a consumer may shave or wax an area of tissue being regulated once or twice in a week or two week period, and apply the personal care composition to the area once or twice on a daily basis. The personal care compositions may be sold separately from acute technologies, or alternatively, be packaged with the acute technologies in the form of a kit, with instructions for using the various components. If sold separately, one or both of the personal care composition and the acute technology may comprise instructions for using the two together according to a regimen for regulating hair growth. Packaging of the personal care composition and the acute technology product may comprise related indicia (e.g., similar, identical, or complementary text, graphics, and/or markings) to provide a visual communication to consumers that the complementary products can be used together to regulate hair growth.

In a second exemplary embodiment, the personal care compositions of the present invention are applied, at alternate times, to areas of the body that may receive other personal care products. For example, a consumer may apply cosmetics to the face or antiperspirants/deodorants to the underarms in the morning, and apply the personal care compositions of the present invention to the same bodily areas in the evening (with or without removal of the other applied product).

The personal care compositions may be offered for sale along with an array of complementary personal care compositions, implements, and/or devices. Methods of marketing the personal care compositions by themselves or together with the complementary compositions, implements, and/or devices are contemplated by the present invention. The methods include communications of using the product or products for hair growth management. The communications can include, for example, text and/or indicia on or in packaging (e.g., product brochures or instructions) or advertisement materials associated with the personal care compositions. Articles of commerce including the personal care compositions and these communications are also provided by the present invention.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-2

Moisturizing skin cream/lotion

Example

| | 1 | 2 |
|---|---|---|
| Component | % w/w | % w/w |
| Polyquaternium 37 | 1.0 | 1.0 |
| Cetylpyridinium chloride | 0.2 | 0.0 |
| Disodium EDTA | 0.1 | 0.1 |
| Glycerine | 7.0 | 7.0 |
| D-Panthenol | 1.0 | 1.0 |
| Sodium Hydroxide | 0.01 | 0.01 |
| Cetearyl Glucoside | 0.2 | 0.2 |
| Ethyl Paraben | 0.2 | 0.2 |
| Propyl Paraben | 0.1 | 0.1 |
| BHT | 0.5 | 0.5 |
| Stearyl alcohol | 0.6 | 0.64 |
| α-phenyl butyl nitrone | 2 | 5 |
| Cetyl alcohol | 0.6 | 0.6 |
| Behenyl Alcohol | 0.4 | 0.4 |
| PEG100 Stearate | 0.1 | 0.1 |
| Polymethylsilsesquioxance | 1.0 | 1.0 |
| Isohexadecane | 3.0 | 3.0 |
| Isopropyl isostearate | 1.5 | 1.5 |
| Sucrose Polycottonseedate | 0.5 | 0.5 |
| DL-alphaTocopheryl acetate | 0.3 | 0.3 |
| Petrolatum | 0.1 | 0.1 |
| Perfume | 0.3 | 0.3 |
| Dimethicone & Dimethiconol | 1.0 | 1.0 |
| Benzyl alcohol | 0.2 | 0.2 |
| Deionised Water | q.s. | q.s. |

### Examples 3-6

Moisturizing skin cream/lotion

| | Example | | | |
|---|---|---|---|---|
| | 3 | 4 | 5 | 6 |
| Component | % w/w | % w/w | % w/w | % w/w |
| Niacinamide | 2.0 | 4.0 | 6.0 | 6.0 |
| Panthenol | 1.0 | 2.0 | 0.5 | 0.5 |
| Polyacrylamide & isoparaffin & | 2.0 | 2.0 | 2.0 | 2.0 |
| laureth-7 | | | | |
| Glycerine | 7 | 7 | 7 | 7 |
| Allantoin | 0.2 | 0.05 | 0.1 | 0.1 |
| Aloe vera gel | 0.1 | 0.1 | 0.1 | 0.1 |
| Tocopheryl acetate | 0.75 | 0.5 | 0.5 | 0.5 |
| Cetyl alcohol | 2.0 | 1.0 | 1.25 | 1.25 |
| Stearyl alcohol | 2.0 | 1.0 | 1.25 | 1.25 |
| Behenyl alcohol | 1.0 | 1.0 | 1.25 | 1.25 |
| Dimethicone & dimethiconol | 0.75 | 0.5 | 0.50 | 0.50 |
| Steareth-21 | 0.6 | 0.4 | 0.5 | 0.5 |
| Steareth-2 | 0.1 | 0.08 | 0.03 | 0.03 |
| PPG-15 stearyl ether | 3.0 | 2.0 | 1.00 | 1.00 |
| Isohexadecane | 0 | 7.0 | 5.0 | 5.0 |
| Hexamidine | 0.1 | 0.3 | 0.1 | 0.3 |
| α-phenyl butyl nitrone | 2 | 2 | 5 | 5 |
| Isononyl isononanoate | 5.0 | 0 | 0 | 5 |
| Dimethicone (350 mm²s⁻¹) | 0.5 | 0.0 | 0.60 | 0.60 |
| Disodium EDTA | 0.10 | 0.10 | 0.10 | 0.10 |
| Nylon 12¹ | 1.5 | 1.0 | 1.1 | 1.1 |
| Titanium Dioxide (and) Mica² | 0.75 | 1.5 | 1.25 | 1.25 |
| BHT | 0.5 | 0.5 | 0.5 | 0.5 |
| Petrolatum | 1.00 | 4.00 | 2.00 | 2.00 |
| Deionised water, fragrance, | q.s. | q.s. | q.s. | q.s. |
| presevatives | | | | |

¹Orgasol ® 2002 D NAT COS.
²A green interference pigment

### Examples 7-11

Antiperspirant soft solid/cream

| | Example | | | |
|---|---|---|---|---|
| | 7 | 8 | 9 | 10 |
| Component | % w/w | % w/w | % w/w | % w/w |
| Al Zr Trichlorohydrex Glycinate | 25 | 25 | 25 | 25 |
| (solid) | | | | |
| Dimethicone (10 cs) | 5.0 | 5.0 | 5.0 | 5.0 |
| Fully Hydrogenated High Erucic | 5.0 | 5.0 | 5.0 | 5.0 |
| Acid Rapeseed oil (HEAR oil) | | | | |
| Hexamidine | 0.1 | 0.3 | 0.1 | 0.3 |
| α-phenyl butyl nitrone | 2 | 2 | 5 | 5 |
| C-18-36 Acid Triglyceride | 1.25 | 1.25 | 1.25 | 1.25 |
| Syncrowax HGLC | | | | |
| Perfume | 0.8 | 0.8 | 0.8 | 0.8 |
| Calcium Pantothenate (solid) | 0.5 | 0 | 3.5 | 0 |
| BHT | 0.5 | 0.5 | 0.5 | 0.5 |
| Tocopherol Acetate | 0.5 | 0 | 0.5 | 0.5 |
| Cyclopentasiloxane | q.s. | q.s. | q.s. | q.s. |

### Example 12-13

Foundation compact

| | Example | |
|---|---|---|
| | 12 | 13 |
| Component | % w/w | % w/w |
| TiO2 silicone treated (SAT treated Tronox CR 837 | 5.0 | 5.0 |
| supplied US Cosmetics) | | |
| Pigment | 1.2 | 1.2 |
| Talc (silicone treated) (Hydrophobic Talc 9742 | 2.3 | 2.3 |
| supplied by Warner Jenkinson) | | |
| hexamidine | 0.1 | 0.1 |
| α-phenyl butyl nitrone | 2 | 5 |
| TiO2-MT100T (micronized TiO2 supplied by Tri-K) | 0.2 | 0.2 |
| DC5225C (dimethicone copolyol - 10% active in | 0.3 | 0.3 |
| cyclomethicone) | | |
| GE SFE 839 Cross-linked Siloxane Elastomer Gel¹ | 48 | 48 |
| Propylparaben (preservative) | 0.10 | 0.10 |
| BHT | 0.5 | 0.5 |
| Glycerine | 7.0 | 7.0 |
| Ozokerite Wax | 3.2 | 3.2 |
| DC245 (cyclomethicone) | q.s. | q.s. |

¹5% Dimethicone/vinyl dimethicone cross-polymer in cyclomethicone

### Examples 14-22

Moisture rinse body wash cleansers

| | Ex. | Ex. | Ex. | Ex. | Ex. | Ex. | Ex. | Ex. | Ex. |
|---|---|---|---|---|---|---|---|---|---|
| | 14 | 15 | 16 | 17 | 18 | 19 | 20 | 21 | 22 |
| Ingredient | wt % | wt % | wt % | wt % | wt % | wt % | wt % | wt % | wt % |
| I. Aqueous Phase | | | | | | | | | |
| Composition | | | | | | | | | |
| Hydroxypropyl Starch | 3.5 | 4.0 | 3.5 | 3.5 | 3.5 | 3.0 | 3.5 | 3.5 | 3.5 |
| Phosphate (Structure XL | | | | | | | | | |
| from National Starch) | | | | | | | | | |
| Emulsifying Wax NF | 2.75 | 3.0 | 2.75 | | 2.75 | 2.5 | | 2.75 | 2.75 |
| (Polawax from Croda) | | | | | | | | | |
| Behenetrimonium | | | | 2.25 | | | 2.0 | | |
| methosulfate and cetearyl | | | | | | | | | |
| alcohol (Incroquat Behenyl | | | | | | | | | |
| TMS from Croda) | | | | | | | | | |
| α-phenyl butyl nitrone | 5.0 | 5.0 | 5.0 | 5.0 | 5.0 | 5.0 | 5.0 | 5.0 | 5.0 |
| Fragrance | 1.0 | 1.0 | 1.0 | 1.0 | 1.0 | 1.0 | 1.0 | 1.0 | 1.0 |
| Preservatives | 0.8 | 0.8 | 0.8 | 0.8 | 0.8 | 0.8 | 0.8 | 0.8 | 0.8 |
| Water | q.s. | q.s. | q.s. | q.s. | q.s. | q.s. | q.s. | q.s. | q.s. |
| II. Lipid/HMIP phase | | | | | | | | | |
| Composition | | | | | | | | | |
| Petrolatum (Superwhite | 15 | | | | 5 | 15 | 15 | 15 | 20 |
| Protopet from WITCO) | | | | | | | | | |
| Mineral Oil (Hydrobrite | | 20 | | | | 2 | | | |
| 1000 PO White MO from | | | | | | | | | |
| WITCO) | | | | | | | | | |
| Dimethicone Fluid (Dow | | | 10 | | | | 5 | | |
| Corning Silicone Fluid | | | | | | | | | |
| 10000 cst) | | | | | | | | | |
| Puresyn 101LT | | | | 15 | | | | | |
| (Polydecene from Exxon | | | | | | | | | |
| Mobile) | | | | | | | | | |
| Sunflower Seed Oil | | | | | 10 | | | 5 | |
| (Lipovol Sun from Lipo) | | | | | | | | | |
| Mica/Titanium | 0.75 | | 1 | | 1.0 | 0.5 | | | |
| Dioxide/Dimethicone | | | | | | | | | |
| (SAT-Timiron MP115 | | | | | | | | | |
| Starluster from US | | | | | | | | | |
| Cosmetics) | | | | | | | | | |
| Titanium | 0.75 | 0.75 | | 2.0 | | | | | |
| Dioxide/Mica/Silica/Dimethicone | | | | | | | | | |
| (SAT-Timiron | | | | | | | | | |
| Splendid Red from US | | | | | | | | | |
| Cosmetics) | | | | | | | | | |
| Mica/Mineral/Titanium | | | | | | 0.5 | | | |
| Dioxide/Iron | | | | | | | | | |
| Oxide/Lecithin (LT- | | | | | | | | | |
| Colorona Red Gold from | | | | | | | | | |
| US Cosmetics) | | | | | | | | | |
| Mica/Titanium | | | | | 0.5 | | | | |
| Dioxide/Dimethicone | | | | | | | | | |
| (SAT-Timiron Super Green | | | | | | | | | |
| from US Cosmetics) | | | | | | | | | |
| Mica/Dimethicone (SA-M- | | | | | | | | | 3.0 |
| M from US Cosmetics | | | | | | | | | |
| Mica/Titanium | | | | | | | 0.5 | | |
| Dioxide/Dimethicone | | | | | | | | | |
| (SAT-Flamenco Ultra Silk | | | | | | | | | |
| 2500 from US Cosmetics) | | | | | | | | | |

### Examples 23-24

Dual phase body wash cleansers

| | Ex 23 | Ex 24 |
|---|---|---|
| Ingredient | wt % | wt % |
| I. Cleansing Phase Composition | | |
| Miracare SLB-365 (from Rhodia) | 47.4 | 47.4 |
| (Sodium Trideceth Sulfate, Sodium | | |
| Lauramphoacetate, Cocamide MEA) | | |
| Guar Hydroxypropyltrimonium Chloride | 0.7 | 0.7 |
| (N-Hance 3196 from Aqualon) | | |
| PEG 90M (Polyox WSR 301 from Dow | 0.2 | 0.2 |
| Chemical) | | |
| α-phenyl butyl nitrone | 5.0 | 5.0 |
| Sodium Chloride | 3.5 | 3.5 |
| Preservatives | 0.84 | 0.84 |
| Citric Acid | 0.4 | 0.4 |
| Perfume | 2.0 | 2.0 |
| Expancel 091 DE 40 d30 (from Expancel, Inc.) | 0.4 | 0.4 |
| Water | q.s. | q.s. |
| (pH) | (6.0) | (6.0) |
| II. Lipid phase Composition | | |
| Petrolatum (Superwhite Protopet from WITCO) | 74.58 | 74.58 |
| Mineral Oil (Hydrobrite 1000 PO White MO | 23.92 | 23.92 |
| from WITCO) | | |
| Mica/Dimethicone | | 1.5 |
| Titanium Dioxide/Mica/Silica/Dimethicone | 1.5 | |
| (SAT-Timiron Splendid Red from US | | |
| Cosmetics) | | |

### Example 25

Body wash cleansers

| Ingredient | wt % |
|---|---|
| I. Phase 1 | |
| Ammonium Laureth-3 Sulfate (25% Active) | 46.7 |
| Citric Acid Anhydrous | 1.76 |
| Sodium Lauroamphoacetate (27%) | 43.47 |
| Trihydroxystearin (Thixcin R from Rheox) | 2.35 |
| α-phenyl butyl nitrone | 5.0 |
| Preservatives | 1.73 |
| Lauric Acid | 2.35 |
| Petrolatum | 1.64 |
| II. Phase 2 | |
| Ammonium Laureth-3 Sulfate | 18 |
| Ammonium Lauryl Sulfate (25% Active) | 12 |
| Phase 1 | 42.6 |
| Fragrance | 1.0 |
| Premix 1 | |
| Guar Hydroxypropyltrimonium Chloride | 0.3 |
| (N-Hance 3196 from Aqualon) | |
| Water | q.s. |
| Premix 2 | |
| Petrolatum | 17.3 |
| Titanium Dioxide/Mica/Silica/Dimethicone | 1.5 |
| (SAT-Timiron Splendid Red from US Cosmetics) | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal-care composition, comprising:
a) a first ingredient selected from the group consisting of α-phenyl butyl nitrone (PBN), PBN doxylcyclohexane radicals, 5,5-dimethyl pyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, spin-trapping derivatives thereof, and mixtures thereof;
b) a second ingredient selected from the group consisting of particulate materials, panthenol, pantothenic acid derivatives, tanning actives, and mixtures thereof, and
c) a dermatologically acceptable carrier.

2. The composition of claim 1, further comprising a third ingredient selected from the group consisting of wherein additional chronic actives are selected from butylated hydroxytoluene, butylated hydroxyanisole, hexamidine, hexyl isobutyrate, menthyl anthranilate, agmatine, aminoguanidine, methofuran, 3-butylidenepthalide, cetyl pyridinium chloride, phytosterols, ursolic acid, catechins, green tea extract, soy peptides, alkane polyols, and mixtures thereof.

3. The composition of claim 1, wherein said composition further comprises at least one additional component selected from the group consisting of hair growth inhibiting compounds, polyquaternium-37, depilatories, desquamation actives, anti-acne actives, anti-wrinkle actives, anti-atrophy actives, anti-oxidant actives, radical scavengers, chelators, anti-inflammatory agents, anti-cellulite agents, topical anesthetics, skin lightening agents, antimicrobial and antifungal actives, sunscreen actives, conditioning agents, thickening agents, and mixtures thereof.

4. The composition of claim 1, wherein the first ingredient comprises α-phenyl butyl nitrone.

5. The composition of claim 1, wherein α-phenyl butyl nitrone is included at level of from about 2% to about 5% by weight of composition.

6. The composition of claim 4, wherein the second ingredient comprises panthenol.

7. A personal care composition, comprising:
a) a first ingredient selected from the group consisting of α-phenyl butyl nitrone (PBN), PBN doxylcyclohexane radicals, 5,5-dimethyl pyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, spin-trapping derivatives thereof, and mixtures thereof,
b) a second ingredient selected from the group consisting of hexamidine, green tea catechins, soy proteins, agmatine, aminoguanidine, and mixtures thereof, and
c) a dermatologically acceptable carrier.

8. The composition of claim 7, wherein the second ingredient comprises hexamidine.

9. The composition of claim 8, further comprising an alkane polyol.

10. The composition of claim 8, further comprising polyquaternium-37.

11. The composition of claim 9, further comprising polyquaternium-37.

11. The composition of claim 7, wherein the first ingredient comprises α-phenyl butyl nitrone.

12. The composition of claim 11, wherein α-phenyl butyl nitrone is included at level of from about 2% to about 5% by weight of composition.

13. A personal care composition, comprising:
a) a first ingredient selected from the group consisting of α-phenyl butyl nitrone (PBN), PBN doxylcyclohexane radicals, 5,5-dimethyl pyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, spin-trapping derivatives thereof, and mixtures thereof, and
b) a dermatologically acceptable carrier,
wherein the composition is in the form of a antiperspirant and/or deodorant product.

14. The composition of claim 13, wherein the first ingredient comprises α-phenyl butyl nitrone.

15. The composition of claim 13, further comprising a material selected from the group consisting of hexamidine, green tea catechins, soy proteins, agmatine, aminoguanidine, butylated hydroxytoluene, and mixtures thereof.

16. A personal care composition, comprising:
a) a first ingredient selected from the group consisting of α-phenyl butyl nitrone (PBN), PBN doxylcyclohexane radicals, 5,5-dimethyl pyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, spin-trapping derivatives thereof, and mixtures thereof; and
b) a dermatologically acceptable carrier,
wherein an amount of the first ingredient is effective for regulating hair growth, and wherein the amount is at least about 2% by weight of the composition.

17. The composition of claim 16, wherein the amount of the first ingredient is from about 2% to about 5% by weight of the composition.

18. The composition of claim 16, wherein the first ingredient comprises α-phenyl butyl nitrone.

19. The composition of claim 16, further comprising other hair growth inhibiting compounds.

20. The composition of claim 16, further comprising at least one additional component selected from the group consisting of hair growth inhibiting compounds, polyquaternium-37, depilatories, desquamation actives, anti-acne actives, anti-wrinkle actives, anti-atrophy actives, anti-oxidant actives, radical scavengers, chelators, anti-inflammatory agents, anti-cellulite agents, topical anesthetics, skin lightening agents, antimicrobial and antifungal actives, sunscreen actives, conditioning agents, thickening agents, and mixtures thereof.
